SEQUENCE LISTING (11 sequences)

SEQ ID NO: 1 - DNA, 1830 bp, Zea mays
  CDS (1)...(1830)
  transit_peptide (1)...(227)
atggcggctctggccacgtcgcagctcgtcgcaacgcgcgccggcctgggcgtcccggacgcgtccacgttccgccgcggcgccgcgcagggcctgagggggggccggacggcgtcggcggcggacacgctcagcatgcggaccagcgcgcgcgcggcgcccaggctccagcaccagcagcagcagcaggcgcgccgcggggccaggttcccgtcgctcgtcgtgtgcgccagcgccggcatgaacgtcgtcttcgtcggcgccgagatggcgccgtggagcaagaccggcggcctcggcgacgtcctcggcggcctgccgccggccatggccgcgaatgggcaccgtgtcatggtcgtctctccccgctacgaccagtacaaggacgcctgggacaccagcgtcgtgtccgagatcaagatgggagacaggtacgagacggtcaggttcttccactgctacaagcgcggagtggaccgcgtgttcgttgaccacccactgttcctggagagggtttggggaaagaccgaggagaagatctacgggcctgacgctggaacggactacagggacaaccagctgcggttcagcctgctatgccaggcagcacttgaagctccaaggatcctgagcctcaacaacaacccatacttctccggaccatacggggaggacgtcgtgttcgtctgcaacgactggcacaccggccctctctcgtgctacctcaagagcaactaccagtcccacggcatctacagggacgcaaagaccgctttctgcatccacaacatctcctaccagggccggttcgccttctccgactacccggagctgaacctcccggagagattcaagtcgtccttcgatttcatcgacggctacgagaagcccgtggaaggccggaagatcaactggatgaaggccgggatcctcgaggccgacagggtcctcaccgtcagcccctactacgccgaggagctcatctccggcatcgccaggggctgcgagctcgacaacatcatgcgcctcaccggcatcaccggcatcgtcaacggcatggacgtcagcgagtgggaccccagcagggacaagtacatcgccgtgaagtacgacgtgtcgacggccgtggaggccaaggcgctgaacaaggaggcgctgcaggcggaggtcgggctcccggtggaccggaacatcccgctggtggcgttcatcggcaggctggaagagcagaagggacccgacgtcatggcggccgccatcccgcagctcatggagatggtggaggacgtgcagatcgttctgctgggcacgggcaagaagaagttcgagcgcatgctcatgagcgccgaggagaagttcccaggcaaggtgcgcgccgtggtcaagttcaacgcggcgctggcgcaccacatcatggccggcgccgacgtgctcgccgtcaccagccgcttcgagccctgcggcctcatccagctgcaggggatgcgatacggaacgccctgcgcctgcgcgtccaccggtggactcgtcgacaccatcatcgaaggcaagaccgggttccacatgggccgcctcagcgtcgactgcaacgtcgtggagccggcggacgtcaagaaggtggccaccaccttgcagcgcgccatcaaggtggtcggcacgccggcgtacgaggagatggtgaggaactgcatgatccaggatctctcctggaagggccctgccaagaactgggagaacgtgctgctcagcctcggggtcgccggcggcgagccaggggtcgaaggcgaggagatcgcgccgctcgccaaggagaacgtggccgcgccctga

SEQ ID NO: 2 - protein, 609 aa, Zea mays
MAALATSQLVATRAGLGVPDASTFRRGAAQGLRGGRTASAADTLSMRTSARAAPRLQHQQQQQARRGARFPSLVVCASAGMNVVFVGAEMAPWSKTGGLGDVLGGLPPAMAANGHRVMVVSPRYDQYKDAWDTSVVSEIKMGDRYETVRFFHCYKRGVDRVFVDHPLFLERVWGKTEEKIYGPDAGTDYRDNQLRFSLLCQAALEAPRILSLNNNPYFSGPYGEDVVFVCNDWHTGPLSCYLKSNYQSHGIYRDAKTAFCIHNISYQGRFAFSDYPELNLPERFKSSFDFIDGYEKPVEGRKINWMKAGILEADRVLTVSPYYAEELISGIARGCELDNIMRLTGITGIVNGMDVSEWDPSRDKYIAVKYDVSTAVEAKALNKEALQAEVGLPVDRNIPLVAFIGRLEEQKGPDVMAAAIPQLMEMVEDVQIVLLGTGKKKFERMLMSAEEKFPGKVRAVVKFNAALAHHIMAGADVLAVTSRFEPCGLIQLQGMRYGTPCACASTGGLVDTIIEGKTGFHMGRLSVDCNVVEPADVKKVATTLQRAIKVVGTPAYEEMVRNCMIQDLSWKGPAKNWENVLLSLGVAGGEPGVEGEEIAPLAKENVAAP

SEQ ID NO: 3 - DNA, 23 bp, Artificial Sequence
  Primer 75360
gatggcggctctggccacgtcgc

SEQ ID NO: 4 - DNA, 22 bp, Artificial Sequence
  Primer 75363
ctcagggcgcggccacgttctc

SEQ ID NO: 5 - DNA, 58 bp, Artificial Sequence
  Primer 75583
gacgtcatggcggccgccatcccgcagctcatggagatggtggaggacgtgcagatcg

SEQ ID NO: 6 - DNA, 29 bp, Artificial Sequence
  Primer 75584
gggatggcggccgccatgacgtcggggcc

SEQ ID NO: 7 - DNA, 30 bp, Artificial Sequence
  Primer 741
gatcgctagctccacggcgccatctcggcg

SEQ ID NO: 8 - DNA, 35 bp, Artificial Sequence
  Primer 742
gatcgtcgactgcagatggcggctctggccacgtc

SEQ ID NO: 9 - DNA, 45 bp, Artificial Sequence
  Primer  739
gatcggatccaccatggtcagggcgcggccacgttctccttggcg

SEQ ID NO: 10 - DNA, 30 bp, Artificial Sequence
  Primer 740
gatcgctagccacatgggccgcctcagcgt

SEQ ID NO: 11 - DNA, 537 bp, Zea mays
  intron (1)...(537): spliceable ADH1 intron
gtccgccttgtttctcctctgtctcttgatctgactaatcttggtttatgattcgttgagtaattttggggaaagcttcgtccacagttttttttcgatgaacagtgccgcagtggcgctgatcttgtatgctatcctgcaatcgtggtgaacttatttcttttatatcctttactcccatgaaaaggctagtaatctttctcgatgtaacatcgtccagcactgctattaccgtgtggtccatccgacagtctggctgaacacatcatacgatctatggagcaaaaatctatcttccctgttctttaatgaaggacgtcattttcattagtatgatctaggaatgttgcaacttgcaaggaggcgtttctttctttgaatttaactaactcgttgagtggccctgtttctcggacgtaaggcctttgctgctccacacatgtccattcgaattttaccgtgtttagcaagggcgaaaagtttgcatcttgatgatttagcttgactatgcgattgctttcctggacccgtgcagct

(19) United States
(12) Patent Application Publication
(10) Pub. No.: US 2007/0261136 A1
(43) Pub. Date: Nov. 8, 2007

(54) High Amylopectin Maize

(75) Inventors: George W. Singletary, Ankeny, IA (US); Lan Zhou, Ankeny, IA (US); Kimberly F. Glassman, Ankeny, IA (US); Douglas M. Haefele, Johnston, IA (US)

Correspondence Address: PIONEER HI-BRED INTERNATIONAL, INC., 7250 N.W. 62ND AVENUE, P.O. BOX 552, JOHNSTON, IA, 50131-0552, US

(73) Assignee: PIONEER HI-BRED INTERNATIONAL, INC., Johnston, IA (US)

(21) Appl. No.: 11/741,821

(22) Filed: Apr. 30, 2007

Related U.S. Application Data
(60) Provisional application No. 60/796,754, filed on May 2, 2006.

(51) Int. Cl. A01H 5/00; C12N 15/82; C12N 5/04

(52) U.S. Cl. 800/284; 800/320.1; 435/412; 435/468

(57) ABSTRACT

The present invention is directed to compositions and methods for producing a dominant high-amylopectin starch trait in cereal grain and methods for screening grain for genetic variation in hydrolysis and fermentation time.

### CROSS-REFERENCE TO RELATED APPLICATIONS

This application claims priority to and the benefit of U.S. Provisional Application No. 60/796,754, filed May 2, 2006, which is herein incorporated in its entirety by reference.

### FIELD OF THE INVENTION

The invention relates to modified starch composition of grain seed. Specifically, the grain is comprised primarily of amylopectin starch and finds utility in numerous industrial, food, and feed applications. Methods for screening grain for genetic variation in hydrolysis and fermentation time are disclosed.

### BACKGROUND OF THE INVENTION

A well-known mutation that alters the starch composition of maize is the waxy mutation, a mutation that inactivates granule bound starch synthase. The mutation causes a reduction in amylose and increase in amylopectin starch in the endosperm of the maize kernel.

A portion of the Dry Grind Ethanol (DGE) industry over the last 2-3 years has been changing the way ethanol is produced from maize grain. In the conventional process, corn is ground to a meal, slurried with water, and “cooked” with thermostable α-amylase at very high temperature to liquify the starch and prepare it for saccharification (i.e., enzymatic hydrolysis of starch-derived dextrins to produce glucose). Released glucose is metabolized by yeast to produce, among other materials, ethanol. In the new process grain is ground and slurried with water, but instead of “cooking” at high temperatures, enzymes (e.g., α-amylase and glucoamylase) are added to release glucose (or oligosaccharides that are converted into glucose) from ungelatinized starch granules. Because starch granules are not gelatinized (i.e., semi-crystalline structure disrupted, allowing dissolution of polymeric carbohydrate), glucose hydrolysis occurs more directly from undisrupted granules. This is commonly referred to as Raw Starch Hydrolysis (RSH) (see. U.S. Patent Publication No. 2004/0234649).

Maize waxy starch, a starch mostly lacking the usual ˜25% amylose and instead containing more nearly 100% amylopectin, hydrolyzes quicker by enzymatic action than ordinary maize starch. Starch hydrolysis in the RSH process occurs more rapidly using maize grain meal containing waxy starch than meal made of regular, amylose-containing starch.

Genetic mutants of maize which produce waxy starch are known but these plants must be homozygous for the dysfunctional gene since the mutation is produced by recessive gene action. Plants (e.g., rice, potato, sweet potato) producing waxy starch have been created by transgenic methods, but these methods have not produced a dominant genotype.

We have demonstrated that transgenic RNAi-produced maize waxy grain releases glucose more rapidly in RSH than corresponding null grain. Plants with this modification can transfer the “waxy trait” in a dominant fashion through cross hybridization. The grain of these plants will allow ethanol to be produced more efficiently in the DGE RSH process

### EMBODIMENTS OF THE INVENTION

The invention is directed to the alteration of starch composition and levels in crop plant seed, resulting in grain with increased amylopectin starch. The claimed sequences encode proteins preferentially expressed during seed development.

Compositions and methods are provided for transgenically producing a dominant, heritable, high amylopectin trait in grain. This is the first known example of a grain of equivalent waxy starch character by this means.

Methods are provided for quickly screening, and collecting data on, many samples to assess variation in hydrolysis and fermentation characteristics.

### DETAILED DESCRIPTION OF THE INVENTION

Compositions of the invention are a novel maize granule-bound starch synthase (GBSS), the enzyme responsible for synthesizing amylose in normal starch; an expression vector comprising a hairpin DNA construct which, when transformed into appropriate grain tissue, elicits RNA interference of GBSS gene expression which functions equivalently to a dominant gene; and maize grain low in amylose and high in amylopectin starch compared to wild type grain and analygous to common waxy starch.

Methods of the invention comprise a method for transgenically producing grain with a dominant waxy (high amylopectin) genotype.

Additionally, the invention includes a method for identifying genetic or transgenic variation in hydrolysis/fermentation time. The method provides a way to quickly screen numerous sources of genetic variation. The method also generates data useful for description of fermentation kinetics. This means data describing the progress of fermentation is collected at many points during the fermentation so that the progress to completion can be described in detail.

### Definitions

Units, prefixes, and symbols may be denoted in their Si accepted form. Unless otherwise indicated, nucleic acids are written left to right in 5′ to 3′ orientation; amino acid sequences are written left to right in amino to carboxy orientation, respectively. Numeric ranges recited within the specification are inclusive of the numbers defining the range and include each integer within the defined range. Amino acids may be referred to herein by either their commonly known three letter symbols or by the one-letter symbols recommended by the IUPAC-IUB Biochemical Nomenclature Commission. Nucleotides, likewise, may be referred to by their commonly accepted single-letter codes. Unless otherwise provided for, software, electrical, and electronics terms as used herein are as defined in The New IEEE Standard Dictionary of Electrical and Electronics Terms (5th edition, 1993). The terms defined below are more fully defined by reference to the specification as a whole.

The term “isolated” refers to material, such as a nucleic acid or a protein, which is: (1) substantially or essentially free from components which normally accompany or interact with the material as found in its naturally occurring environment or (2) if the material is in its natural environment, the material has been altered by deliberate human intervention to a composition and/or placed at a locus in the cell other than the locus native to the material.

As used herein, the term “nucleic acid” means a polynucleotide and includes single or multi-stranded polymers of deoxyribonucleotide or ribonucleotide bases. Nucleic acids may also include fragments and modified nucleotides. Therefore, as used herein, the terms “nucleic acid” and “polynucleotide” are used interchangably.

As used herein, “polypeptide” means proteins, protein fragments, modified proteins (e.g., glycosylated, phosphorylated, or other modifications), amino acid sequences and synthetic amino acid sequences. The polypeptide can be modified or not. Therefore, as used herein, “polypeptide” and “protein” are used interchangably.

As used herein, “plant” includes plants and plant parts including but not limited to plant cells and plant tissues such as leaves, stems, roots, flowers, pollen, and seeds.

As used herein, “promoter” includes reference to a region of DNA upstream from the start of transcription and involved in recognition and binding of RNA polymerase and other proteins to initiate transcription.

By “fragment” is intended a portion of the nucleotide sequence or a portion of the amino acid sequence and hence protein encoded thereby. Fragments of a nucleotide sequence may encode protein fragments that retain the biological activity of the native nucleic acid, functional fragments. Alternatively, fragments of a nucleotide sequence that can be useful as hybridization probes may not encode fragment proteins retaining biological activity. Thus, fragments of a nucleotide sequence are generally greater than 25, 50, 100, 150, 200, 250, 300, 350, 400, 450, 500, 600, or 700 nucleotides and up to and including the entire nucleotide sequence encoding the proteins of the invention. Generally the probes are less than 1000 nucleotides and often less than 500 nucleotides. Fragments of the invention include antisense sequences used to decrease expression of the inventive polynucleotides. Such antisense fragments may vary in length ranging from greater than 25, 50, 100, 200, 300, 400, 500, 600, or 700 nucleotides and up to and including the entire coding sequence.

By “variants” is intended substantially similar sequences. Generally, nucleic acid sequence variants of the invention will have at least 60%, 65%, 70%, 75%, 76%, 77%, 78%, 79%, 80%, 85%, 86%, 87%, 88%, 89%, 90%, 91%, 92%, 93%, 94%, 95%, 96%, 97%, 98% or 99% sequence identity to the native nucleotide sequence, wherein the % sequence identity is based on the entire sequence and is determined by GAP 10 analysis using default parameters. Generally, polypeptide sequence variants of the invention will have at least about 60%, 65%, 70%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, or 99% sequence identity to the native protein, wherein the % sequence identity is based on the entire sequence and is determined by GAP 10 analysis using default parameters. GAP uses the algorithm of Needleman and Wunsch (J. Mol. Biol. 48:443-453, 1970) to find the alignment of two complete sequences that maximizes the number of matches and minimizes the number of gaps.

As used herein “stable transformation” refers to the transfer of a nucleic acid fragment into a genome of a host organism (this includes both nuclear and organelle genomes) resulting in genetically stable inheritance. In addition to traditional methods, stable transformation includes the alteration of gene expression by any means including chimeraplasty or transposon insertion.

As used herein “transient transformation” refers to the transfer of a nucleic acid fragment or protein into the nucleus (or DNA-containing organelle) of a host organism resulting in gene expression without integration and stable inheritance.

As used herein “transformation” may include stable transformation and transient transformation. Unless otherwise stated, “transformation” refers to stable transformation.

Typically, “grain” means the mature kernel produced by commercial growers for purposes other than growing or reproducing the species, and “seed” means the mature kernel used for growing or reproducing the species. For the purposes of the present invention, “grain”, “seed”, and “kernel”, will be used interchangeably.

As used herein, “genetically modified” or “genetically altered” means the modified expression of a seed protein resulting from one or more genetic modifications; the modifications including but not limited to: recombinant gene technologies, and breeding stably genetically modified plants to produce progeny comprising the altered gene product.

Methods of the invention involve increasing or inhibiting a seed protein by such means as, but are not limited to, transgenic expression, antisense suppression, co-suppression methods including but not limited to: RNA interference, gene activation or suppression using transcription factors and/or repressors, mutagenesis including transposon tagging, directed and site-specific mutagenesis, chromosome engineering (see Nobrega et. al., Nature 431:988-993(04)), homologous recombination, TILLING (Targeting Induced Local Lesions In Genomes), and biosynthetic competition to manipulate, in plants and plant seeds and grains, the expression of seed proteins, including, but not limited to, those encoded by the sequences disclosed herein.

Transgenic plants producing seeds and grain with altered seed protein content are also provided.

The genetically modified seed and grain of the invention can also be obtained by breeding with transgenic plants, by breeding between independent transgenic events, and by breeding of transgenic plants with plants with one or more alleles of genes encoding GBSS. Breeding, including introgression of transgenic loci into elite breeding germplasm and adaptation (improvement) of breeding germplasm to the expression of transgenes, can be facilitated by methods such as by marker assisted selected breeding.

### Nucleic Acids

The isolated nucleic acids of the present invention can be made using (a) standard recombinant methods, (b) synthetic techniques, or combinations thereof. In some embodiments, the polynucleotides of the present invention can be cloned, amplified, or otherwise constructed from a monocot or dicot. Typical examples of monocots are corn, sorghum, barley, wheat, millet, rice, or turf grass. Typical dicots include soybeans, safflower, sunflower, canola, alfalfa, potato, or cassava.

Functional fragments included in the invention can be obtained using primers which selectively hybridize under stringent conditions. Primers are generally at least 12 bases in length and can be as high as 200 bases, but will generally be from 15 to 75, or more likely from 15 to 50 bases. Functional fragments can be identified using a variety of techniques such as restriction analysis, Southern analysis, primer extension analysis, and DNA sequence analysis.

The present invention includes a plurality of polynucleotides that encode for the identical amino acid sequence. The degeneracy of the genetic code allows for such “silent variations” which can be used, for example, to selectively hybridize and detect allelic variants of polynucleotides of the present invention. Additionally, the present invention includes isolated nucleic acids comprising allelic variants. The term “allele” as used herein refers to a related nucleic acid of the same gene.

Variants of nucleic acids included in the invention can be obtained, for example, by oligonucleotide-directed mutagenesis, linker-scanning mutagenesis, mutagenesis using the polymerase chain reaction, and the like. See, for example, pages 8.0.3-8.5.9 Current Protocols in Molecular Biology, Ausubel et al., Eds., Greene Publishing and Wiley-Interscience, New York (1995). Also, see generally, McPherson (ed.), DIRECTED MUTAGENESIS: A Practical Approach, (IRL Press, 1991). Thus, the present invention also encompasses DNA molecules comprising nucleotide sequences that have substantial sequence similarity with the inventive sequences.

Variants included in the invention may contain individual substitutions, deletions or additions to the nucleic acid or polypeptide sequences which alter, add or delete a single amino acid or a small percentage of amino acids in the encoded sequence. A “conservatively modified variant” is an alteration which results in the substitution of an amino acid with a chemically similar amino acid. When the nucleic acid is prepared or altered synthetically, advantage can be taken of known codon preferences of the intended host.

With respect to particular nucleic acid sequences, conservatively modified variants refers to those nucleic acids which encode identical or conservatively modified variants of the amino acid sequences. Because of the degeneracy of the genetic code, a large number of functionally identical nucleic acids encode any given protein. For instance, the codons GCA, GCC, GCG and GCU all encode the amino acid alanine. Thus, at every position where an alanine is specified by a codon, the codon can be altered to any of the corresponding codons described without altering the encoded polypeptide. Such nucleic acid variations are “silent variations” and represent one species of conservatively modified variation. Every nucleic acid sequence herein that encodes a polypeptide also, by reference to the genetic code, describes every possible silent variation of the nucleic acid. One of ordinary skill will recognize that each codon in a nucleic acid (except AUG, which is ordinarily the only codon for methionine; and UGG, which is ordinarily the only codon for tryptophan) can be modified to yield a functionally identical molecule. Accordingly, each silent variation of a nucleic acid which encodes a polypeptide of the present invention is implicit in each described polypeptide sequence and is within the scope of the claimed invention.

As to amino acid sequences, one of skill will recognize that individual substitutions, deletions or additions to a nucleic acid, peptide, polypeptide, or protein sequence which alters, adds or deletes a single amino acid or a small percentage of amino acids in the encoded sequence is a “conservatively modified variant” where the alteration results in the substitution of an amino acid with a chemically similar amino acid. Thus, any number of amino acid residues selected from the group of integers consisting of from 1 to 15 can be so altered. Thus, for example, 1, 2, 3, 4, 5, 7, or 10 alterations can be made. Conservatively modified variants typically provide similar biological activity as the unmodified polypeptide sequence from which they are derived. For example, substrate specificity, enzyme activity, or ligand/receptor binding is generally at least 30%, 40%, 50%, 60%, 70%, 80%, or 90% of the native protein for its native substrate. Conservative substitution tables providing functionally similar amino acids are well known in the art.

For example, the following six groups each contain amino acids that are conservative substitutions for one another:

### 1) Alanine (A), Serine (S), Threonine (T);

### 2) Aspartic acid (D), Glutamic acid (E);

### 3) Asparagine (N), Glutamine (Q);

### 4) Arginine (R), Lysine (K);

### 5) Isoleucine (I), Leucine (L), Methionine (M), Valine (V); and

### 6) Phenylalanine (F), Tyrosine (Y), Tryptophan (W).

See also, Creighton (1984) Proteins W.H. Freeman and Company, other acceptable conservative substitution patterns known in the art may also be used, such as the scoring matrices of sequence comparison programs like the GCG package, BLAST, or CLUSTAL for example.

The claimed invention also includes “shufflents” produced by sequence shuffling of the inventive polynucleotides to obtain a desired characteristic. Sequence shuffling is described in PCT publication No. 96/19256. See also, Zhang, J. H., et al., Proc. Natl. Acad. Sci. USA 94:4504-4509 (1997).

The present invention also includes the use of 5′ and/or 3′ UTR regions for modulation of translation of heterologous coding sequences. Positive sequence motifs include translational initiation consensus sequences (Kozak, Nucleic Acids Res. 15:8125 (1987)) and the 7-methylguanosine cap structure (Drummond et al., Nucleic Acids Res. 13:7375 (1985)). Negative elements include stable intramolecular 5′ UTR stem-loop structures (Muesing et al., Cell 48:691 (1987)) and AUG sequences or short open reading frames preceded by an appropriate AUG in the 5′ UTR (Kozak, supra, Rao et al., Mol. Cell. Biol. 8:284 (1988)).

Further, the polypeptide-encoding segments of the polynucleotides of the present invention can be modified to alter codon usage. Altered codon usage can be employed to alter translational efficiency. Codon usage in the coding regions of the polynucleotides of the present invention can be analyzed statistically using commercially available software packages such as “Codon Preference” available from the University of Wisconsin Genetics Computer Group (see Devereaux et al., Nucleic Acids Res. 12:387-395 (1984)) or MacVector 4.1 (Eastman Kodak Co., New Haven, Conn.).

For example, the inventive nucleic acids can be optimized for enhanced expression in plants of interest. See, for example, Perlak et al. (1991) Proc. Natl. Acad. Sci. USA 88:3324-3328; and Murray et al. (1989) Nucleic Acids Res. 17:477-498, the disclosure of which is incorporated herein by reference. In this manner, the polynucleotides can be synthesized utilizing plant-preferred codons.

The present invention provides subsequences comprising isolated nucleic acids containing at least 20 contiguous bases of the claimed sequences. For example the isolated nucleic acid includes those comprising at least 20, 30, 40, 50, 60, 70, 80, 90, 100, 200, 300, 400, 500, 600, 700 or 800 contiguous nucleotides of the claimed sequences. Subsequences of the isolated nucleic acid can be used to modulate or detect gene expression by introducing into the subsequences compounds which bind, intercalate, cleave and/or crosslink to nucleic acids.

The nucleic acids of the claimed invention may conveniently comprise a multi-cloning site comprising one or more endonuclease restriction sites inserted into the nucleic acid to aid in isolation of the polynucleotide. Also, translatable sequences may be inserted to aid in the isolation of the translated polynucleotide of the present invention. For example, a hexa-histidine marker sequence, or a GST fusion sequence, provides a convenient means to purify the proteins of the claimed invention.

A polynucleotide of the claimed invention can be attached to a vector, adapter, promoter, transit peptide or linker for cloning and/or expression of a polynucleotide of the present invention. Additional sequences may be added to such cloning and/or expression sequences to optimize their function in cloning and/or expression, to aid in isolation of the polynucleotide, or to improve the introduction of the polynucleotide into a cell. Use of cloning vectors, expression vectors, adapters, and linkers is well known and extensively described in the art.

For a description of such nucleic acids see, for example, Stratagene Cloning Systems, Catalogs 1995, 1996, 1997 (La Jolla, Calif.); and, Amersham Life Sciences, Inc, Catalog '97 (Arlington Heights, Ill.).

The isolated nucleic acid compositions of this invention, such as RNA, cDNA, genomic DNA, or a hybrid thereof, can be obtained from plant biological sources using any number of cloning methodologies known to those of skill in the art. In some embodiments, oligonucleotide probes which selectively hybridize, under stringent conditions, to the polynucleotides of the present invention are used to identify the desired sequence in a cDNA or genomic DNA library.

Exemplary total RNA and mRNA isolation protocols are described in Plant Molecular Biology: A Laboratory Manual, Clark, Ed., Springer-Verlag, Berlin (1997); and, Current Protocols in Molecular Biology, Ausubel et al., Eds., Greene Publishing and Wiley-Interscience, New York (1995). Total RNA and mRNA isolation kits are commercially available from vendors such as Stratagene (La Jolla, Calif.), Clonetech (Palo Alto, Calif.), Pharmacia (Piscataway, N.J.), and 5′-3′ (Paoli, Pa.). See also, U.S. Pat. Nos. 5,614,391; and, 5,459,253.

Typical cDNA synthesis protocols are well known to the skilled artisan and are described in such standard references as: Plant Molecular Biology: A Laboratory Manual, Clark, Ed., Springer-Verlag, Berlin (1997); and, Current Protocols in Molecular Biology, Ausubel et al., Eds., Greene Publishing and Wiley-Interscience, New York (1995). cDNA synthesis kits are available from a variety of commercial vendors such as Stratagene or Pharmacia.

An exemplary method of constructing a greater than 95% pure full-length cDNA library is described by Carninci et al., Genomics 37:327-336 (1996). Other methods for producing full-length libraries are known in the art. See, e.g., Edery et al., Mol. Cell Biol. 15(6):3363-3371 (1995); and PCT Application WO 96/34981. It is often convenient to normalize a cDNA library to create a library in which each clone is more equally represented. A number of approaches to normalize cDNA libraries are known in the art. Construction of normalized libraries is described in Ko, Nucl. Acids. Res. 18(19):5705-5711 (1990); Patanjali et al., Proc. Natl. Acad. U.S.A. 88:1943-1947 (1991); U.S. Pat. Nos. 5,482,685 and 5,637,685; and Soares et al., Proc. Natl. Acad. Sci. USA 91:9228-9232 (1994).

Subtracted cDNA libraries are another means to increase the proportion of less abundant cDNA species. See, Foote et al. in, Plant Molecular Biology: A Laboratory Manual, Clark, Ed., Springer-Verlag, Berlin (1997); Kho and Zarbl, Technique 3(2):58-63 (1991); Sive and St. John, Nucl. Acids Res. 16(22):10937 (1988); Current Protocols in Molecular Biology, Ausubel et al., Eds., Greene Publishing and Wiley-Interscience, New York (1995); and, Swaroop et al., Nucl. Acids Res. 19(8):1954 (1991). cDNA subtraction kits are commercially available. See, e.g., PCR-Select (Clontech).

To construct genomic libraries, large segments of genomic DNA are generated by random fragmentation. Examples of appropriate molecular biological techniques and instructions are found in Sambrook et al., Molecular Cloning: A Laboratory Manual, 2nd Ed., Cold Spring Harbor Laboratory, Vols. 1-3 (1989), Methods in Enzymology, Vol. 152: Guide to Molecular Cloning Techniques, Berger and Kimmel, Eds., San Diego: Academic Press, Inc. (1987), Current Protocols in Molecular Biology, Ausubel et al., Eds., Greene Publishing and Wiley-Interscience, New York (1995); Plant Molecular Biology: A Laboratory Manual, Clark, Ed., Springer-Verlag, Berlin (1997). Kits for construction of genomic libraries are also commercially available.

The cDNA or genomic library can be screened using a probe based upon the sequence of a nucleic acid of the present invention such as those disclosed herein. Probes may be used to hybridize with genomic DNA or cDNA sequences to isolate homologous polynucleotides in the same or different plant species. Those of skill in the art will appreciate that various degrees of stringency of hybridization can be employed in the assay; and either the hybridization or the wash medium can be stringent. The degree of stringency can be controlled by temperature, ionic strength, pH and the presence of a partially denaturing solvent such as formamide.

Typically, stringent hybridization conditions will be those in which the salt concentration is less than about 1.5 M Na ion, typically about 0.01 to 1.0 M Na ion concentration (or other salts) at pH 7.0 to 8.3 and the temperature is at least about 30° C. for short probes (e.g., 10 to 50 nucleotides) and at least about 60° C. for long probes (e.g., greater than 50 nucleotides). Stringent conditions may also be achieved with the addition of destabilizing agents such as formamide.

Exemplary low stringency conditions include hybridization with a buffer solution of 30 to 35% formamide, 1 M NaCl, 1% SDS (sodium dodecyl sulfate) at 37° C., and a wash in 1× to 2×SSC (20×SSC=3.0 M NaCl/0.3 M trisodium citrate) at 50° C. Exemplary moderate stringency conditions include hybridization in 40 to 45% formamide, 1 M NaCl, 1% SDS at 37° C., and a wash in 0.5× to 1×SSC at 55° C. Exemplary high stringency conditions include hybridization in 50% formamide, 1 M NaCl, 1% SDS at 37° C., and a wash in 0.1×SSC at 60° C. Typically the time of hybridization is from 4 to 16 hours.

An extensive guide to the hybridization of nucleic acids is found in Tijssen, Laboratory Techniques in Biochemistry and Molecular Biology—Hybridization with Nucleic Acid Probes, Part I, Chapter 2 “Overview of principles of hybridization and the strategy of nucleic acid probe assays”, Elsevier, New York (1993); and Current Protocols in Molecular Biology, Chapter 2, Ausubel et al., Eds., Greene Publishing and Wiley-Interscience, New York (1995). Often, cDNA libraries will be normalized to increase the representation of relatively rare cDNAs.

The following terms are used to describe the sequence relationships between two or more nucleic acids or polynucleotides: (a) “reference sequence”, (b) “comparison window”, (c) “sequence identity”, (d) “percentage of sequence identity”, and (e) “substantial identity”.

(a) As used herein, “reference sequence” is a defined sequence used as a basis for sequence comparison. A reference sequence may be a subset or the entirety of a specified sequence; for example, as a segment of a full-length cDNA or gene sequence, or the complete cDNA or gene sequence.

(b) As used herein, “comparison window” makes reference to a contiguous and specified segment of a polynucleotide sequence, wherein the polynucleotide sequence in the comparison window may comprise additions or deletions (i.e., gaps) compared to the reference sequence (which does not comprise additions or deletions) for optimal alignment of the two sequences. Generally, the comparison window is at least 20 contiguous nucleotides in length, and optionally can be 30, 40, 50, 100, or longer. Those of skill in the art understand that to avoid a high similarity to a reference sequence due to inclusion of gaps in the polynucleotide sequence a gap penalty is typically introduced and is subtracted from the number of matches.

Methods of alignment of sequences for comparison are well known in the art. Thus, the determination of percent sequence identity between any two sequences can be accomplished using a mathematical algorithm. Non-limiting examples of such mathematical algorithms are the algorithm of Myers and Miller (1988) CABIOS 4:11-17; the local homology algorithm of Smith et al. (1981) Adv. Appl. Math. 2:482; the homology alignment algorithm of Needleman and Wunsch (1970) J. Mol. Biol. 48:443-453; the search-for-similarity-method of Pearson and Lipman (1988) Proc. Natl. Acad. Sci. 85:2444-2448; the algorithm of Karlin and Altschul (1990) Proc. Natl. Acad. Sci. USA 872264, modified as in Karlin and Altschul (1993) Proc. Natl. Acad. Sci. USA 90:5873-5877.

Computer implementations of these mathematical algorithms can be utilized for comparison of sequences to determine sequence identity. Such implementations include, but are not limited to: CLUSTAL in the PC/Gene program (available from Intelligenetics, Mountain View, Calif.); the ALIGN program (Version 2.0) and GAP, BESTFIT, BLAST, FASTA, and TFASTA in the Wisconsin Genetics Software Package, Version 8 (available from Genetics Computer Group (GCG), 575 Science Drive, Madison, Wis., USA). Alignments using these programs can be performed using the default parameters. The CLUSTAL program is well described by Higgins et al. (1988) Gene 73:237-244 (1988); Higgins et al. (1989) CABIOS 5:151-153; Corpet et al. (1988) Nucleic Acids Res. 16:10881-90; Huang et al. (1992) CABIOS 8:155-65; and Pearson et al. (1994) Meth. Mol. Biol. 24:307-331. The ALIGN program is based on the algorithm of Myers and Miller (1988) supra. A PAM120 weight residue table, a gap length penalty of 12, and a gap penalty of 4 can be used with the ALIGN program when comparing amino acid sequences. The BLAST programs of Altschul et al (1990) J. Mol. Biol. 215:403 are based on the algorithm of Karlin and Altschul (1990) supra. BLAST nucleotide searches can be performed with the BLASTN program, score=100, wordlength=12, to obtain nucleotide sequences homologous to a nucleotide sequence encoding a protein of the invention. BLAST protein searches can be performed with the BLASTX program, score=50, wordlength=3, to obtain amino acid sequences homologous to a protein or polypeptide of the invention. To obtain gapped alignments for comparison purposes, Gapped BLAST (in BLAST 2.0) can be utilized as described in Altschul et al. (1997) Nucleic Acids Res. 25:3389. Alternatively, PSI-BLAST (in BLAST 2.0) can be used to perform an iterated search that detects distant relationships between molecules. See Altschul et al. (1997) supra. When utilizing BLAST, Gapped BLAST, PSI-BLAST, the default parameters of the respective programs (e.g., BLASTN for nucleotide sequences, BLASTX for proteins) can be used. Alignment may also be performed manually by inspection.

Unless otherwise stated, sequence identity/similarity values provided herein refer to the value obtained using GAP version 10 using the following parameters: % identity using GAP Weight of 50 and Length Weight of 3; % similarity using Gap Weight of 12 and Length Weight of 4, or any equivalent program, aligned over the full length of the sequence. By “equivalent program” is intended any sequence comparison program that, for any two sequences in question, generates an alignment having identical nucleotide or amino acid residue matches and an identical percent sequence identity when compared to the corresponding alignment generated by GAP Version 10.

GAP uses the algorithm of Needleman and Wunsch (1970) J. Mol. Biol. 48:443-453, to find the alignment of two complete sequences that maximizes the number of matches and minimizes the number of gaps. GAP considers all possible alignments and gap positions and creates the alignment with the largest number of matched bases and the fewest gaps. It allows for the provision of a gap creation penalty and a gap extension penalty in units of matched bases. GAP must make a profit of gap creation penalty number of matches for each gap it inserts. If a gap extension penalty greater than zero is chosen, GAP must, in addition, make a profit for each gap inserted of the length of the gap times the gap extension penalty. Default gap creation penalty values and gap extension penalty values in Version 10 of the Wisconsin Genetics Software Package for protein sequences are 8 and 2, respectively. For nucleotide sequences the default gap creation penalty is 50 while the default gap extension penalty is 3. The gap creation and gap extension penalties can be expressed as an integer selected from the group of integers consisting of from 0 to 200. Thus, for example, the gap creation and gap extension penalties can be 0, 1, 2, 3, 4, 5, 6, 7, 8, 9, 10, 15, 20, 25, 30, 35, 40, 45, 50, 55, 60, 65 or greater.

GAP presents one member of the family of best alignments. There may be many members of this family, but no other member has a better quality. GAP displays four figures of merit for alignments: Quality, Ratio, Identity, and Similarity. The Quality is the metric maximized in order to align the sequences. Ratio is the quality divided by the number of bases in the shorter segment. Percent Identity is the percent of the symbols that actually match. Percent Similarity is the percent of the symbols that are similar. Symbols that are across from gaps are ignored. A similarity is scored when the scoring matrix value for a pair of symbols is greater than or equal to 0.50, the similarity threshold. The scoring matrix used in Version 10 of the Wisconsin Genetics Software Package is BLOSUM62 (see Henikoff and Henikoff (1989) Proc. Natl. Acad. Sci. USA 89:10915).

(c) As used herein, “sequence identity” or “identity” in the context of two nucleic acid or polypeptide sequences makes reference to the residues in the two sequences that are the same when aligned for maximum correspondence over a specified comparison window. When percentage of sequence identity is used in reference to proteins it is recognized that residue positions which are not identical often differ by conservative amino acid substitutions, where amino acid residues are substituted for other amino acid residues with similar chemical properties (e.g., charge or hydrophobicity) and therefore do not change the functional properties of the molecule. When sequences differ in conservative substitutions, the percent sequence identity may be adjusted upwards to correct for the conservative nature of the substitution. Sequences that differ by such conservative substitutions are said to have “sequence similarity” or “similarity”. Means for making this adjustment are well known to those of skill in the art. Typically this involves scoring a conservative substitution as a partial rather than a full mismatch, thereby increasing the percentage sequence identity. Thus, for example, where an identical amino acid is given a score of 1 and a non-conservative substitution is given a score of zero, a conservative substitution is given a score between zero and 1. The scoring of conservative substitutions is calculated, e.g., as implemented in the program PC/GENE (Intelligenetics, Mountain View, Calif.).

(d) As used herein, “percentage of sequence identity” means the value determined by comparing two optimally aligned sequences over a comparison window, wherein the portion of the polynucleotide sequence in the comparison window may comprise additions or deletions (i.e., gaps) as compared to the reference sequence (which does not comprise additions or deletions) for optimal alignment of the two sequences. The percentage is calculated by determining the number of positions at which the identical nucleic acid base or amino acid residue occurs in both sequences to yield the number of matched positions, dividing the number of matched positions by the total number of positions in the window of comparison, and multiplying the result by 100 to yield the percentage of sequence identity.

(e)(i) The term “substantial identity” of polynucleotide sequences means that a polynucleotide comprises a sequence that has at least 60%, 65%, 70%, 75%, 80%, 85%, 90%, 91%, 92%, 93%, 94%, 95%, 96%, 97%, 98%, or 99% or more sequence identity compared to a reference sequence using one of the alignment programs described using standard parameters. One of skill in the art will recognize that these values can be appropriately adjusted to determine corresponding identity of proteins encoded by two nucleotide sequences by taking into account codon degeneracy, amino acid similarity, reading frame positioning, and the like. Substantial identity of amino acid sequences for these purposes normally means sequence identity of at least 60%, 65%, 70%, 75%, 80%, 85%, 90%, 91%, 92%, 93%, 94%, 95%, 96%, 97%, 98%, or 99% or more.

Another indication that nucleotide sequences are substantially identical is if two molecules hybridize to each other under stringent conditions. Generally, stringent conditions are selected to be about 5° C. lower than the thermal melting point (Tm) for the specific sequence at a defined ionic strength and pH. However, stringent conditions encompass temperatures in the range of about 1° C. to about 20° C. lower than the Tm, depending upon the desired degree of stringency as otherwise qualified herein. Nucleic acids that do not hybridize to each other under stringent conditions are still substantially identical if the polypeptides they encode are substantially identical. This may occur, e.g., when a copy of a nucleic acid is created using the maximum codon degeneracy permitted by the genetic code. One indication that two nucleic acid sequences are substantially identical is when the polypeptide encoded by the first nucleic acid is immunologically cross reactive with the polypeptide encoded by the second nucleic acid.

(e)(ii) The term “substantial identity” in the context of a peptide indicates that a peptide comprises a sequence with at least 60%, 65%, 70%, 75%, 80%, 85%, 90%, 91%, 92%, 93%, 94%, 95%, 96%, 97%, 98%, or 99% or more sequence identity to the reference sequence over a specified comparison window. Alignment can be conducted using the homology alignment algorithm of Needleman and Wunsch (1970) J. Mol. Biol. 48:443-453. An indication that two peptide sequences are substantially identical is that one peptide is immunologically reactive with antibodies raised against the second peptide. Peptides that are “substantially similar” comprise a sequence with at least 60%, 65%, 70%, 75%, 80%, 85%, 90%, 91%, 92%, 93%, 94%, 95%, 96%, 97%, 98%, or 99% or more sequence identity or sequence similarity to the reference sequence over a specified comparison window. In this case residue positions that are not identical instead differ by conservative amino acid changes.

The nucleic acids of the invention can be amplified from nucleic acid samples using amplification techniques. For instance, polymerase chain reaction (PCR) technology can be used to amplify the sequences of polynucleotides of the present invention and related polynucleotides directly from genomic DNA or cDNA libraries. PCR and other in vitro amplification methods may also be useful, for example, to clone nucleic acid sequences that code for proteins to be expressed, to make nucleic acids to use as probes for detecting the presence of the desired mRNA in samples, for nucleic acid sequencing, or for other purposes.

Examples of techniques useful for in vitro amplification methods are found in Berger, Sambrook, and Ausubel, as well as Mullis et al., U.S. Pat. No. 4,683,202 (1987); and, PCR Protocols A Guide to Methods and Applications, Innis et al., Eds., Academic Press Inc., San Diego, Calif. (1990). Commercially available kits for genomic PCR amplification are known in the art. See, e.g., Advantage-GC Genomic PCR Kit (Clontech). The T4 gene 32 protein (Boehringer Mannheim) can be used to improve yield of long PCR products. PCR-based screening methods have also been described. Wilfinger et al. describe a PCR-based method in which the longest cDNA is identified in the first step so that incomplete clones can be eliminated from study. BioTechniques, 22(3):481-486 (1997).

In one aspect of the invention, nucleic acids can be amplified from a plant nucleic acid library. The nucleic acid library may be a cDNA library, a genomic library, or a library generally constructed from nuclear transcripts at any stage of intron processing. Libraries can be made from a variety of plant tissues such as ears, seedlings, leaves, stalks, roots, pollen, or seeds. Good results have been obtained using tissues such as night-harvested earshoot with husk at stage V-12 from corn line B73, corn night-harvested leaf tissue at stage V8-V10 from line B73, corn anther tissue at prophase I from line B73, 4 DAP coenocytic embryo sacs from corn line B73, 67 day old corn cob from corn line L, and corn BMS suspension cells treated with chemicals related to phosphatases.

Alternatively, the sequences of the invention can be used to isolate corresponding sequences in other organisms, particularly other plants, more particularly, other monocots. In this manner, methods such as PCR, hybridization, and the like can be used to identify such sequences having substantial sequence similarity to the sequences of the invention. See, for example, Sambrook et al. (1989) Molecular Cloning: A Laboratory Manual (2nd ed., Cold Spring Harbor Laboratory Press, Plainview, N.Y.) and Innis et al. (1990), PCR Protocols: A Guide to Methods and Applications (Academic Press, New York). Coding sequences isolated based on their sequence identity to the entire inventive coding sequences set forth herein or to fragments thereof are encompassed by the present invention.

The isolated nucleic acids of the present invention can also be prepared by direct chemical synthesis by methods such as the phosphotriester method of Narang et al., Meth. Enzymol. 68:90-99 (1979); the phosphodiester method of Brown et al., Meth. Enzymol. 68:109-151 (1979); the diethylphosphoramidite method of Beaucage et al., Tetra. Lett. 22:1859-1862 (1981); the solid phase phosphoramidite triester method described by Beaucage and Caruthers, Tetra. Lett. 22(20):1859-1862 (1981), e.g., using an automated synthesizer, e.g., as described in Needham-VanDevanter et al., Nucleic Acids Res. 12:6159-6168 (1984); and, the solid support method of U.S. Pat. No. 4,458,066. Chemical synthesis generally produces a single stranded oligonucleotide. This may be converted into double stranded DNA by hybridization with a complementary sequence, or by polymerization with a DNA polymerase using the single strand as a template. One of skill will recognize that while chemical synthesis of DNA is limited to sequences of about 100 bases, longer sequences may be obtained by the ligation of shorter sequences.

### Expression Cassettes

In another embodiment expression cassettes comprising isolated nucleic acids of the present invention are provided. An expression cassette will typically comprise a polynucleotide of the present invention operably linked to transcriptional initiation regulatory sequences which will direct the transcription of the polynucleotide in the intended host cell, such as tissues of a transformed plant.

By “operably linked” is intended a functional linkage between a nucleic acid sequence and a subsequent sequence. Generally, in the context of an expression cassette, operably linked means that the nucleotide sequences being linked are contiguous and, where necessary to join two or more protein coding regions, contiguous and in the same reading frame. In the case where an expression cassette contains two or more protein coding regions joined in a contiguous manner in the same reading frame, the polynucleotide is herein referred to as a chimeric polynucleotide, nucleic acid or fragment. The cassette may additionally contain at least one additional coding sequence in sense or antisense orientation to be co-transformed into the organism. An intron sequence can be added to the 5′ untranslated region or to the coding sequence or the partial coding sequence. See for example Buchman and Berg, Mol. Cell Biol. 8:4395-4405 (1988); Callis et al., Genes Dev. 1:1183-1200 (1987). Use of maize introns Adh1-S intron 1, 2, and 6, the Bronze-1 intron are known in the art. See generally, The Maize Handbook, Chapter 116, Freeling and Walbot, Eds., Springer, New York (1994).

Alternatively, the additional coding sequence(s) can be provided on multiple expression cassettes.

The construction of such expression cassettes which can be employed in conjunction with the present invention is well known to those of skill in the art in light of the present disclosure. See, e.g., Sambrook et al.; Molecular Cloning: A Laboratory Manual; Cold Spring Harbor, N.Y.; (1989); Gelvin et al.; Plant Molecular Biology Manual (1990); Plant Biotechnology: Commercial Prospects and Problems, eds. Prakash et al.; Oxford & IBH Publishing Co.; New Delhi, India; (1993); and Heslot et al.; Molecular Biology and Genetic Engineering of Yeasts; CRC Press, Inc., USA; (1992); each incorporated herein in its entirety by reference.

For example, plant expression vectors may include (1) a cloned plant gene under the transcriptional control of 5′ and 3′ regulatory sequences and (2) a dominant selectable marker. Such plant expression vectors may also contain, if desired, a promoter regulatory region (e.g., one conferring inducible, constitutive, environmentally- or developmentally-regulated, or cell- or tissue-specific/selective expression), a transcription initiation start site, a ribosome binding site, an RNA processing signal, a transcription termination site, and/or a polyadenylation signal.

Constitutive, tissue-preferred or inducible promoters can be employed. Examples of constitutive promoters include the cauliflower mosaic virus (CaMV) 35S transcription initiation region, the 1′- or 2′-promoter derived from T-DNA of Agrobacterium tumefaciens, the actin promoter, the ubiquitin promoter, the histone H₂B promoter (Nakayama et al., 1992, FEBS Lett 30:167-170), the Smas promoter, the cinnamyl alcohol dehydrogenase promoter (U.S. Pat. No. 5,683,439), the Nos promoter, the pEmu promoter, the rubisco promoter, the GRP1-8 promoter, and other transcription initiation regions from various plant genes known in the art.

Examples of inducible promoters are the Adh1 promoter which is inducible by hypoxia or cold stress, the Hsp70 promoter which is inducible by heat stress, the PPDK promoter which is inducible by light, the In2 promoter which is safener induced, the ERE promoter which is estrogen induced and the pepcarboxylase promoter which is light induced.

Examples of promoters under developmental control include promoters that initiate transcription preferentially in certain tissues, such as leaves, roots, fruit, pollen, seeds, or flowers. An exemplary promoter is the anther specific promoter 5126 (U.S. Pat. Nos. 5,689,049 and 5,689,051). Examples of seed-preferred promoters include, but are not limited to, 27 kD gamma zein promoter and waxy promoter, (Boronat, A., et al., Plant Sci. 47:95-102 (1986); Reina, M., et al., Nucleic Acids Res. 18(21):6426 (1990); Kloesgen, R. B., et al., Mol. Gen. Genet. 203:237-244 (1986)), as well as the globulin 1, oleosin and the phaseolin promoters. The disclosures each of these are incorporated herein by reference in their entirety.

The barley or maize Nucl promoter, the maize Cim1 promoter or the maize LTP2 promoter can be used to preferentially express in the nucellus. See, for example WO 00/11177, the disclosure of which is incorporated herein by reference.

Either heterologous or non-heterologous (i.e., endogenous) promoters can be employed to direct expression of the nucleic acids of the present invention. These promoters can also be used, for example, in expression cassettes to drive expression of sense nucleic acids or antisense nucleic acids to reduce, increase, or alter concentration and/or composition of the proteins of the present invention in a desired tissue.

If polypeptide expression is desired, it is generally desirable to include a polyadenylation region at the 3′-end of a polynucleotide coding region. The polyadenylation region can be derived from the natural gene, from a variety of other plant genes, or from T-DNA. The 3′ end sequence to be added can be derived from, for example, the nopaline synthase or octopine synthase genes, or alternatively from another plant gene, or less preferably from any other eukaryotic gene.

The vector comprising the sequences from a polynucleotide of the present invention will typically comprise a marker gene which confers a selectable phenotype on plant cells. Usually, the selectable marker gene encodes antibiotic or herbicide resistance. Suitable genes include those coding for resistance to the antibiotics spectinomycin and streptomycin (e.g., the aada gene), the streptomycin phosphotransferase (SPT) gene coding for streptomycin resistance, the neomycin phosphotransferase (NPTII) gene encoding kanamycin or geneticin resistance, the hygromycin phosphotransferase (HPT) gene coding for hygromycin resistance.

Suitable genes coding for resistance to herbicides include those which act to inhibit the action of acetolactate synthase (ALS), in particular the sulfonylurea type herbicides (e.g., the acetolactate synthase (ALS) gene containing mutations leading to such resistance in particular the S4 and/or Hra mutations), those which act to inhibit action of glutamine synthase, such as phosphinothricin or basta (e.g., the bar gene), or other such genes known in the art. The bar gene encodes resistance to the herbicide basta and the ALS gene encodes resistance to the herbicide chlorsulfuron.

Typical vectors useful for expression of genes in higher plants are well known in the art and include vectors derived from the tumor-inducing (Ti) plasmid of Agrobacterium tumefaciens described by Rogers et al., Meth. In Enzymol. 153:253-277 (1987). Exemplary A. tumefaciens vectors useful herein are plasmids pKYLX6 and pKYLX7 of Schardl et al., Gene 61:1-11 (1987) and Berger et al., Proc. Natl. Acad. Sci. USA 86:8402-8406 (1989). Another useful vector herein is plasmid pBI101.2 that is available from Clontech Laboratories, Inc. (Palo Alto, Calif.).

A variety of plant viruses that can be employed as vectors are known in the art and include cauliflower mosaic virus (CaMV), geminivirus, brome mosaic virus, and tobacco mosaic virus.

A polynucleotide of the claimed invention can be expressed in either sense or anti-sense orientation as desired. In plant cells, it has been shown that antisense RNA inhibits gene expression by preventing the accumulation of mRNA which encodes the enzyme of interest, see, e.g., Sheehy et al., Proc. Natl. Acad. Sci. USA 85:8805-8809 (1988); and Hiatt et al., U.S. Pat. No. 4,801,340.

Another method of suppression is sense suppression. Introduction of nucleic acid configured in the sense orientation has been shown to be an effective means by which to block the transcription of target genes. For an example of the use of this method to modulate expression of endogenous genes see, Napoli et al., The Plant Cell 2:279-289 (1990) and U.S. Pat. No. 5,034,323. Recent work has shown suppression with the use of double stranded RNA. Such work is described in Tabara et al., Science 282:5388:430-431 (1998). Hairpin approaches of gene suppression are disclosed in WO 98/53083 and WO 99/53050.

Catalytic RNA molecules or ribozymes can also be used to inhibit expression of plant genes. The inclusion of ribozyme sequences within antisense RNAs confers RNA-cleaving activity upon them, thereby increasing the activity of the constructs. The design and use of target RNA-specific ribozymes is described in Haseloff et al., Nature 334:585-591 (1988).

A variety of cross-linking agents, alkylating agents and radical generating species as pendant groups on polynucleotides of the present invention can be used to bind, label, detect, and/or cleave nucleic acids. For example, Vlassov, V. V., et al., Nucleic Acids Res (1986) 14:4065-4076, describe covalent bonding of a single-stranded DNA fragment with alkylating derivatives of nucleotides complementary to target sequences. A report of similar work by the same group is that by Knorre, D. G., et al., Biochimie (1985) 67:785-789. Iverson and Dervan also showed sequence-specific cleavage of single-stranded DNA mediated by incorporation of a modified nucleotide which was capable of activating cleavage (J. Am. Chem. Soc. (1987) 109:1241-1243). Meyer, R. B., et al., J. Am. Chem. Soc. (1989) 111:8517-8519, effect covalent crosslinking to a target nucleotide using an alkylating agent complementary to the single-stranded target nucleotide sequence. A photoactivated crosslinking to single-stranded oligonucleotides mediated by psoralen was disclosed by Lee, B. L., et al., Biochemistry (1988) 27:3197-3203. Use of crosslinking in triple-helix forming probes was also disclosed by Home et al., J. Am. Chem. Soc. (1990) 112:2435-2437. Use of N4, N4-ethanocytosine as an alkylating agent to crosslink to single-stranded oligonucleotides has also been described by Webb and Matteucci, J. Am. Chem. Soc. (1986) 108:2764-2765; Nucleic Acids Res (1986) 14:7661-7674; Feteritz et al., J. Am. Chem. Soc. 113:4000 (1991). Various compounds to bind, detect, label, and/or cleave nucleic acids are known in the art. See, for example, U.S. Pat. Nos. 5,543,507; 5,672,593; 5,484,908; 5,256,648; and 5,681941.

### Gene or Trait Stacking

In certain embodiments the nucleic acid sequences of the present invention can be combined with any combination of polynucleotide sequences of interest or mutations in order to create plants with a desired phenotype. The combinations generated can also include multiple copies of any one of the polynucleotides of interest.

The polynucleotides of the present invention can also be combined with any other gene or combination of genes to produce plants with a variety of desired trait combinations including, but not limited to, traits desirable for animal feed such as high oil genes (e.g., U.S. Pat. No. 6,232,529); balanced amino acids (e.g. hordothionins (U.S. Pat. Nos. 5,990,389; 5,885,801; 5,885,802; 5,703,409 and 6,800,726); high lysine (Williamson et al. (1987) Eur. J. Biochem. 165:99-106; and WO 98/20122); and high methionine proteins (Pedersen et al. (1986) J. Biol. Chem. 261:6279; Kirihara et al. (1988) Gene 71:359; and Musumura et al. (1989) Plant Mol. Biol. 12: 123)); and thioredoxins (U.S. application Ser. No. 10/005,429, filed Dec. 3, 2001)), the disclosures of which are herein incorporated by reference. The polynucleotides of the present invention can also be combined with traits desirable for insect, disease or herbicide resistance (e.g., Bacillus thuringiensis toxic proteins (U.S. Pat. Nos. 5,366,892; 5,747,450; 5,737,514; 5723,756; 5,593,881; Geiser et al. (1986) Gene 48:109); lectins (Van Damme et al. (1994) Plant Mol. Biol. 24:825); fumonisin detoxification genes (U.S. Pat. No. 5,792,931); avirulence and disease resistance genes (Jones et al. (1994) Science 266:789; Martin et al. (1993) Science 262:1432; Mindrinos et al. (1994) Cell 78:1089); acetolactate synthase (ALS) mutants that lead to herbicide resistance such as the S4 and/or Hra mutations; inhibitors of glutamine synthase such as phosphinothricin or basta (e.g., bar gene); and glyphosate resistance (EPSPS gene)); and traits desirable for processing or process products such as high oil (e.g., U.S. Pat. No. 6,232,529); modified oils (e.g., fatty acid desaturase genes (U.S. Pat. No. 5,952,544; WO 94/11516)); modified starches (e.g., ADPG pyrophosphorylases (AGPase), starch synthases (SS), starch branching enzymes (SBE) and starch debranching enzymes (SDBE)); and polymers or bioplastics (e.g., U.S. Pat. No. 5,602,321; beta-ketothiolase, polyhydroxybutyrate synthase, and acetoacetyl-CoA reductase (Schubert et al. (1988) J. Bacteriol. 170:5837-5847), the disclosures of which are herein incorporated by reference.

One can also combine the polynucleotides of the present invention with polynucleotides providing agronomic traits such as male sterility (e.g., see U.S. Pat. No. 5,583,210), stalk strength, flowering time, or transformation technology traits such as cell cycle regulation or gene targeting (e.g. WO 99/61619; WO 00/17364; WO 99/25821), the disclosures of which are herein incorporated by reference.

These combinations can be created by any method including, but not limited to, cross breeding plants by any conventional or TopCross methodology, by homologous recombination, site specific recombination, or other genetic modification. If the traits are combined by genetically transforming the plants, the polynucleotide sequences of interest can be combined at any time and in any order. For example, a transgenic plant comprising one or more desired traits can be used as the target to introduce further traits by subsequent transformation. The traits can be introduced simultaneously in a co-transformation protocol with the polynucleotides of interest provided by any combination of transformation cassettes. For example, if two sequences will be introduced, the two sequences can be contained in separate transformation cassettes (trans) or contained on the same transformation cassette (cis). Expression of the sequences can be driven by the same promoter or by different promoters. In certain cases, it may be desirable to introduce a transformation cassette that will suppress the expression of the polynucleotide of interest. This may be combined with any combination of other suppression cassettes or overexpression cassettes to generate the desired combination of traits in the plant. Traits may also be combined by transformation and mutation by any known method.

### Transformation of Cells

The method of transformation is not critical to the present invention; various methods of transformation are currently available. As newer methods are available to transform crops or other host cells they may be directly applied. Accordingly, a wide variety of methods have been developed to insert a DNA sequence into the genome of a host cell to obtain the transcription and/or translation of the sequence to effect phenotypic changes in the organism. Thus, any method which provides for efficient transformation/transfection may be employed.

A DNA sequence coding for the desired polynucleotide of the present invention, for example a cDNA or a genomic sequence encoding a full length protein, can be used to construct an expression cassette which can be introduced into the desired plant. Isolated nucleic acid acids of the present invention can be introduced into plants according to techniques known in the art. Generally, expression cassettes as described above and suitable for transformation of plant cells are prepared.

Techniques for transforming a wide variety of higher plant species are well known and described in the technical, scientific, and patent literature. See, for example, Weising et al., Ann. Rev. Genet. 22:421-477 (1988). For example, the DNA construct may be introduced directly into the genomic DNA of the plant cell using techniques such as electroporation, PEG poration, particle bombardment, silicon fiber delivery, or microinjection of plant cell protoplasts or embryogenic callus. See, e.g., Tomes et al., Direct DNA Transfer into Intact Plant Cells Via Microprojectile Bombardment. pp. 197-213 in Plant Cell, Tissue and Organ Culture, Fundamental Methods, Eds. O. L. Gamborg and G. C. Phillips, Springer-Verlag Berlin Heidelberg New York, 1995. Alternatively, the DNA constructs may be combined with suitable T-DNA flanking regions and introduced into a conventional Agrobacterium tumefaciens host vector. The virulence functions of the Agrobacterium tumefaciens host will direct the insertion of the construct and adjacent marker into the plant cell DNA when the cell is infected by the bacteria. See, U.S. Pat. No. 5,591,616.

The introduction of DNA constructs using polyethylene glycol precipitation is described in Paszkowski et al., Embo J. 3:2717-2722 (1984). Electroporation techniques are described in Fromm et al., Proc. Natl. Acad. Sci. U.S.A. 82:5824 (1985). Ballistic transformation techniques are described in Klein et al., Nature 327:70-73 (1987).

Agrobacterium tumefaciens-meditated transformation techniques are well described in the scientific literature. See, for example Horsch et al., Science 233:496-498 (1984), and Fraley et al., Proc. Natl. Acad. Sci. 80:4803 (1983). For instance, Agrobacterium transformation of maize is described in U.S. Pat. No. 5,981,840. Agrobacterium transformation of soybean is described in U.S. Pat. No. 5,563,055.

Other methods of transformation include (1) Agrobacterium rhizogenes-mediated transformation (see, e.g., Lichtenstein and Fuller In: Genetic Engineering, Vol. 6, P. W. J. Rigby, Ed., London, Academic Press, 1987; and Lichtenstein, C. P. and Draper, J. In: DNA Cloning, Vol. 11, D. M. Glover, Ed., Oxford, IR1 Press, 1985), Application PCT/US87/02512 (WO 88/02405 published Apr. 7, 1988) describes the use of A. rhizogenes strain A4 and its R1 plasmid along with A. tumefaciens vectors pARC8 or pARC16, (2) liposome-mediated DNA uptake (see, e.g., Freeman et al., Plant Cell Physiol. 25:1353 (1984)), and (3) the vortexing method (see, e.g., Kindle, Proc. Natl. Acad. Sci. USA 87:1228 (1990)).

DNA can also be introduced into plants by direct DNA transfer into pollen as described by Zhou et al., Methods in Enzymology 101:433 (1983); D. Hess, Intern Rev. Cytol., 107:367 (1987); Luo et al., Plant Mol. Biol. Reporter 6:165 (1988). Expression of polypeptide coding polynucleotides can be obtained by injection of the DNA into reproductive organs of a plant as described by Pena et al., Nature 325:274 (1987). DNA can also be injected directly into the cells of immature embryos and the rehydration of desiccated embryos as described by Neuhaus et al., Theor. Appl. Genet. 75:30 (1987); and Benbrook et al., in Proceedings Bio Expo 1986, Butterworth, Stoneham, Mass., pp. 27-54 (1986).

Animal and lower eukaryotic (e.g., yeast) host cells are competent or rendered competent for transformation by various means. There are several well-known methods of introducing DNA into animal cells. These include: calcium phosphate precipitation, fusion of the recipient cells with bacterial protoplasts containing the DNA, treatment of the recipient cells with liposomes containing the DNA, DEAE dextran, electroporation, biolistics, and micro-injection of the DNA directly into the cells. The transfected cells are cultured by means well known in the art. Kuchler, R. J., Biochemical Methods in Cell Culture and Virology, Dowden, Hutchinson and Ross, Inc. (1977).

### Transgenic Plant Regeneration

Transformed plant cells which are derived by any of the above transformation techniques can be cultured to regenerate a whole plant which possesses the transformed genotype. Such regeneration techniques often rely on manipulation of certain phytohormones in a tissue culture growth medium, typically relying on a biocide and/or herbicide marker that has been introduced together with a polynucleotide of the present invention. For transformation and regeneration of maize see, Gordon-Kamm et al., The Plant Cell 2:603-618 (1990).

Plants cells transformed with a plant expression vector can be regenerated, e.g., from single cells, callus tissue or leaf discs according to standard plant tissue culture techniques. It is well known in the art that various cells, tissues, and organs from almost any plant can be successfully cultured to regenerate an entire plant. Plant regeneration from cultured protoplasts is described in Evans et al., Protoplasts Isolation and Culture, Handbook of Plant Cell Culture, Macmillan Publishing Company, New York, pp. 124-176 (1983); and Binding, Regeneration of Plants, Plant Protoplasts, CRC Press, Boca Raton, pp. 21-73 (1985).

The regeneration of plants containing the foreign gene introduced by Agrobacterium can be achieved as described by Horsch et al., Science, 227:1229-1231 (1985) and Fraley et al., Proc. Natl. Acad. Sci. U.S.A. 80:4803 (1983). This procedure typically produces shoots within two to four weeks and these transformant shoots are then transferred to an appropriate root-inducing medium containing the selective agent and an antibiotic to prevent bacterial growth. Transgenic plants of the present invention may be fertile or sterile.

Regeneration can also be obtained from plant callus, explants, organs, or parts thereof. Such regeneration techniques are described generally in Klee et al., Ann. Rev. Plant Phys. 38:467-486 (1987). The regeneration of plants from either single plant protoplasts or various explants is well known in the art. See, for example, Methods for Plant Molecular Biology, A. Weissbach and H. Weissbach, eds., Academic Press, Inc., San Diego, Calif. (1988). For maize cell culture and regeneration see generally, The Maize Handbook, Freeling and Walbot, Eds., Springer, New York (1994); Corn and Corn Improvement, 3rd edition, Sprague and Dudley Eds., American Society of Agronomy, Madison, Wis. (1988).

One of skill will recognize that after the expression cassette is stably incorporated in transgenic plants and confirmed to be operable, it can be introduced into other plants by sexual crossing. Any of a number of standard breeding techniques can be used, depending upon the species to be crossed.

In vegetatively propagated crops, mature transgenic plants can be propagated by the taking of cuttings, via production of apomictic seed, or by tissue culture techniques to produce multiple identical plants. Selection of desirable transgenics is made and new varieties are obtained and propagated vegetatively for commercial use. In seed propagated crops, mature transgenic plants can be self crossed to produce a homozygous inbred plant. The inbred plant produces seed containing the newly introduced heterologous nucleic acid. These seeds can be grown to produce plants that would produce the selected phenotype.

Parts obtained from the regenerated plant, such as flowers, seeds, leaves, branches, fruit, and the like are included in the invention, provided that these parts comprise cells comprising the isolated nucleic acid of the present invention. Progeny and variants, and mutants of the regenerated plants are also included within the scope of the invention, provided that these parts comprise the introduced nucleic acid sequences.

Transgenic plants expressing a selectable marker can be screened for transmission of the nucleic acid of the present invention by, for example, standard immunoblot and DNA detection techniques. Transgenic lines are also typically evaluated on levels of expression of the heterologous nucleic acid. Expression at the RNA level can be determined initially to identify and quantitate expression-positive plants. Standard techniques for RNA analysis can be employed and include PCR amplification assays using oligonucleotide primers designed to amplify only the heterologous RNA templates and solution hybridization assays using heterologous nucleic acid-specific probes. The RNA-positive plants can then be analyzed for protein expression by Western immunoblot analysis using the specifically reactive antibodies of the present invention. In addition, in situ hybridization and immunocytochemistry according to standard protocols can be done using heterologous nucleic acid specific polynucleotide probes and antibodies, respectively, to localize sites of expression within transgenic tissue. Generally, a number of transgenic lines are usually screened for the incorporated nucleic acid to identify and select plants with the most appropriate expression profiles.

The present invention provides a method of genotyping a plant comprising a polynucleotide of the present invention. Genotyping provides a means of distinguishing homologs of a chromosome pair and can be used to differentiate segregants in a plant population. Molecular marker methods can be used for phylogenetic studies, characterizing genetic relationships among crop varieties, identifying crosses or somatic hybrids, localizing chromosomal segments affecting monogenic traits, map based cloning, and the study of quantitative inheritance. See, e.g., Plant Molecular Biology: A Laboratory Manual, Chapter 7, Clark, Ed., Springer-Verlag, Berlin (1997). For molecular marker methods, see generally, The DNA Revolution by Andrew H. Paterson 1996 (Chapter 2) in: Genome Mapping in Plants (ed. Andrew H. Paterson) by Academic Press/R. G. Landis Company, Austin, Tex., pp. 7-21.

The particular method of genotyping in the present invention may employ any number of molecular marker analytic techniques such as, but not limited to, restriction fragment length polymorphisms (RFLPs). RFLPs are the product of allelic differences between DNA restriction fragments caused by nucleotide sequence variability. Thus, the present invention further provides a means to follow segregation of a gene or nucleic acid of the present invention as well as chromosomal sequences genetically linked to these genes or nucleic acids using such techniques as RFLP analysis.

Plants which can be used in the method of the invention include, but are not limited to, monocotyledons. Preferred plants include maize, wheat, rice, barley, oats, sorghum, millet, or rye.

Seeds derived from plants regenerated from transformed plant cells, plant parts or plant tissues, or progeny derived from the regenerated transformed plants, may be used directly as feed or food, or industrial processes such as dry grind ethanol production.

### Screening for Fermentation Efficiency

The present invention also provides a method for screening plants for relative efficiency in hydrolysis/fermentation. In North American starch-based fuel ethanol production, the time required for starch hydrolysis and fermentation is commonly between 60 and 80 hours. This is a relatively long hydrolysis/fermentation time when compared to ethanol production from simple sugars (e.g. sucrose from sugarcane.) Production costs would be reduced if hydrolysis/fermentation times could be reduced (improved capital utilization and volumetric efficiency.)

Even within a crop type, e.g. corn, it is known that the genotype of the plant material being processed, among other factors, influences fermentation time

In order identify genetic variation in hydrolysis/fermentation time it was necessary to identify a method that allowed us to quickly screen numerous sources of genetic variation. The method was also required to generate data that was useful for description of fermentation kinetics. This means collection of data describing the progress of fermentation at many time points so progress to completion can be described in detail.

The method used to make these measurements is a novel adaptation of the principles and equipment used originally for quantification of differences in forage digestibility for animal nutrition. (See Schofield P. et al, 1995, J. Dairy Sci 78:2230-2238; Schofield P. et al, 1994, J. Anim. Sci. 72:2980-2991; and Pell A. N., et al, 1993, J Dairy Sci 76:1063-1073).

The system is based on the principle that under the normal conditions found in a yeast-catalyzed ethanolic fermentation one mole of glucose is fermented by the yeast to produce two moles of ethanol and two moles of carbon dioxide. Since an equimolar amount of ethanol and carbon dioxide is necessarily produced, the quantification of carbon dioxide produced allows calculation of the amount of ethanol produced. See Example 4. The method thus provides a way to screen crops for fermentation efficiency.

All publications cited in this application are herein incorporated by reference to the same extent as if each individual publication or patent application was specifically and individually indicated to be incorporated by reference.

The present invention will be further described by reference to the following detailed examples. It is understood, however, that there are many extensions, variations, and modifications on the basic theme of the present invention beyond that shown in the examples and description, which are within the spirit and scope of the present invention.

### EXAMPLES

### Example 1

### Isolation of Gene Conferring Waxy Phenotype

A total of six full-length ESTs of PCO295352 and one full-length EST of PCO297360 were obtained and fully sequenced. The sequencing data showed these clones were not full-length. They were all truncated at the same position near the C-terminus of the WAXY protein. Interestingly, the missing portion was found in a different contig (PCO229438). An attempt to clone a full-length waxy from 22 DAP B73 endosperm by RT-PCR was unsuccessful, demonstrating the difficulty of isolating the full length coding region.

The full-length coding region consisting of a transit peptide and a mature protein was constructed by ligating two fragments via PCR: one fragment (from the first amino acid methionine to the amino acid #419 alanine) was from the EST clone p0020.cdenh96r and the other (from the amino acid #422 to the translation stop codon) was from the EST clone p0034.cdnaa95r. The missing amino acids #420 and #421 were introduced by the PCR primers (PHN75583 and 75584). These amino acids were deduced from public sequences of maize GBSS.

p0020.cdenh96r=B73 “Endosperm” “ ” “11 DAP Endosperm dissected”
p0034.cdnaa95r=B73 “Endosperm” “ ” “35 DAP endosperm”

PHN75360:

(23mers, SEQ ID NO: 3)

GATGGCGGCTCTGGCCACGTCGC
PHN75363:

(22mers, SEQ ID NO: 4)

CTCAGGGCGCGGCCACGTTCTC
PHN75583:

(58mers, SEQ ID NO: 5)

GACGTCATGGCGGCCGCCATCCCGCAGCTCATGGAGATGGTGGAGGACGT
GC
AGATCG
PHN75584:

(29mers, SEQ ID NO: 6)

GGGATGGCGGCCGCCATGACGTCGGGGCC

### PCR Conditions:

(a) Reaction A (Roche's Tgo Kit)

| | |
|---|---|
| Vol = 50 ul | |
| 5X PCR Buffer | 10 ul |
| dNTP (10 mM ea) | 1 ul |
| Tgo | 1 ul |
| dH2O | 35 ul |
| p0020.cdenh96r (20 ng/ul) | 1 ul |
| PHN75360 (10 uM) | 1 ul |
| PHN75584 (10 uM) | 1 ul |

9\tilde{4}° C. 2 min ---→ 9\tilde{4}° C. 30 sec --→ 5\tilde{5}° C. 1 min --→
7\tilde{2}° C. 1.5 min --→ 7\tilde{2}° C. 10 min
30 Cycles

(b) Reaction B (Roche's Tgo Kit)

| | |
|---|---|
| Vol = 50 ul | |
| 5X PCR Buffer | 10 ul |
| dNTP (10 mM ea) | 1 ul |
| Tgo | 1 ul |
| dH2O | 35 ul |
| p0034.cdnaa95r (20 ng/ul) | 1 ul |
| PHN75363 (10 uM) | 1 ul |
| PHN75583 (10 uM) | 1 ul |

9\tilde{4}° C. 2 min ---→ 9\tilde{4}° C. 30 sec --→ 5\tilde{5}° C. 1 min -→
7\tilde{2}° C. 1.5 min -→ 7\tilde{2}° C. 10 min
30 Cycles

(c) Reaction C (Roche's Expend High Fidelity Kit)

| | |
|---|---|
| Vol = 50 ul | |
| 10X PCR Buffer with MgCl2 | 5 ul |
| dNTP (10 mM ea) | 1 ul |
| Polymerase | 1 ul |
| dH2O | 39 ul |
| 1.2 Kb PCR Product of Reaction A (50 ng) | 1 ul |
| 0.6 Kb PCR Product of Reaction B (50 ng) | 1 ul |
| PHN75360 (10 uM) | 1 ul |
| PHN75363 (10 uM) | 1 ul |

9\tilde{4}° C. 2 min ---→ 9\tilde{4}° C. 30 sec --→ 5\tilde{5}° C. 1 min -→
7\tilde{2}° C. 1.5 min -→ 7\tilde{2}° C. 10 min
30 Cycles

The 1.8 Kb PCR Product of Reaction C was isolated from agarose gel and subcloned into pCR4.0-TOPO (Invitrogen™). The resulting clone (PHP22499) was confirmed by restriction enzyme digests and sequencing of the entire insert on both strands and submitted for vector construction.

### Example 2

### Vector Construction

A vector for silencing the endogenous waxy gene of maize was constructed as follows: two regions of the WAXY coding sequence were identified as having the least homology with other starch synthases. The first region comprised nt 1-nt 280 of the waxy coding sequence (SEQ ID NO:1) including the transit peptide sequence and was PCR-cloned using the following primer pairs:

| | |
|---|---|
| TMS741: | |
| GATCGCTAGCTCCACGGCGCCATCTCGGCG | (SEQ ID NO: 7) |
| and | |
| TMS742: | |
| GATCGTCGACTGCAGATGGCGGCTCTGGCCACGTC | (SEQ ID NO: 8) |

The second fragment (nt1561-nt1827 of the waxy coding sequence, SEQ ID NO:1) was PCR-cloned using the following primer pairs:

TMS739:

(SEQ ID NO: 9)

GATCGGATCCACCATGGTCAGGGCGCGGCCACGTTCTCCTTGGCG
TMS740:

(SEQ ID NO: 10)

GATCGCTAGCCACATGGGCCGCCTCAGCGT

All four primers also served to add convenient restriction sites to both ends of each fragment. The PCR fragments were ligated together to form a 556 bp chimeric waxy fragment in the pSPORT (BRL) cloning vector.

This plasmid was digested separately with two sets of flanking restriction sites and the two resulting chimeric waxy fragments were used in a four-piece ligation with a restriction fragment containing the spliceable ADH1 INTRON1 of Z. mays (SEQ ID NO:11, or see also: Callis, J., Fromm, M., and V. Walbot; Genes & Development, Vol 1, 1183-1200, 1987 or Luehrsen, K R, and Walbot, V.; 1994. Genes Dev 8:1117-1130) and a compatibly-digested backbone plasmid comprising the GZW64A promoter flanked by ATT L1 and ATT L2 Gateway™ recombinational cloning sequences. Restriction sites were chosen to preferentially generate a cassette with the GZW64A promoter (Reina, M. et al, (1990), Nucleic Acids Res., 18:6426) driving an inverted repeat of the chimeric waxy fragment with the spliceable ADH1 intron inserted between the two arms of the inverted repeat.

Transformed E. coli colonies were confirmed by restriction digest analysis of isolated plasmid DNA. The plasmid was designated PHP23457. The waxy silencing cassette was then introduced by single-site recombinational cloning (Gateway™ and Clonase™) into a binary vector comprising an herbicide-resistance selectable marker to generate PHP23473. The herbicide-resistance selectable marker cassette contained the following elements operably linked, in order: the ubiquitin promoter (Christensen et al. (1989) Plant Mol. Biol. 12:619-632 and Christensen et al. (1992) Plant Mol. Biol. 18:675-689), maize ubiquitin intron (GenBank Accession No. S94464), maize optimized PAT (Wohlleben et al. (1988) Gene 70:25-37), PinII terminator (An et al., Plant Cell 1:115-122, 1989). This plasmid was subsequently introduced into Agrobacterium tumefaciens (LBA4404) carrying the superbinary vector PHP10523 (Japan Tobacco) and the resulting cointegrate (PHP23504) was used in Agrobacterium-mediated transformation of Z. mays.

A vector for simultaneously down-regulating the endogenous waxy gene of maize and overexpressing the brittle1 gene of maize for smaller starch granules was constructed as follows: two restriction fragments comprising the chimeric fragment of WAXY coding sequence described in the previous example were similarly isolated and ligated into an inverted repeat (with ADH1 INTRON1) construct, with the exception that the promoter in the Gateway™ plasmid backbone was CZ19D1 promoter (19 kd alpha zein D1, see U.S. Pat. No. 6,225,529). The resulting plasmid was designated PHP23458.

The over-expression cassette for BRITTLE1 was created by ligating the coding sequence for BRITTLE1 between the GZW64A promoter and terminator flanked by ATT L4 and ATT R1 Gateway™ recombination sites. This plasmid (PHP22822) was included in a multisite Gateway™ recombination reaction with PHP23458, PHP20770 (comprising the herbicide-resistance selectable marker cassette described above) and a binary destination vector (PHP20640) to generate PHP23497. This plasmid was subsequently introduced into Agrobacterium tumefaciens (LBA4404) carrying the superbinary vector PHP10523 (Japan Tobacco) and the resulting cointegrate (PHP23505) was used in Agrobacterium-mediated transformation of Z. mays.

### Example 3

### Agrobacterium Mediated Transformation of Maize

For Agrobacterium-mediated transformation of maize, an expression cassette of the present invention was constructed and the method of Zhao was employed (U.S. Pat. No. 5,981,840, and PCT patent publication WO98/32326; the contents of which are hereby incorporated by reference). Briefly, immature embryos were isolated from maize and the embryos contacted with a suspension of Agrobacterium, where the bacteria are capable of transferring the nucleotide sequence of interest to at least one cell of at least one of the immature embryos (step 1: the infection step). In this step the immature embryos were immersed in an Agrobacterium suspension for the initiation of inoculation. The embryos were co-cultured for a time with the Agrobacterium (step 2: the co-cultivation step). The immature embryos were cultured on solid medium following the infection step. Following this co-cultivation period an optional “resting” step was performed. In this resting step, the embryos were incubated in the presence of at least one antibiotic known to inhibit the growth of Agrobacterium without the addition of a selective agent for plant transformants (step 3: resting step). The immature embryos were cultured on solid medium with antibiotic, but without a selecting agent, for elimination of Agrobacterium and for a resting phase for the infected cells. Next, inoculated embryos were cultured on medium containing a selective agent and the growing transformed callus was recovered (step 4: the selection step). The immature embryos were cultured on solid medium with a selective agent resulting in the selective growth of transformed cells. The callus was then regenerated into plants (step 5: the regeneration step), and calli grown on selective medium were cultured on solid medium to regenerate the plants.

### Example 4

### aIV Screening Process

A sample (e.g. 0.5 grams) of ground plant material (e.g. corn, sorghum grain) was added to a reaction vessel (e.g. a 125 ml serum vial) containing 25 ml of a suitable aqueous buffer chosen to maintain the pH of the reaction mixture close to the pH recommended by the manufacturer of the starch hydrolyzing enzyme(s) being used (e.g. 100 mM citric acid-dibasic sodium phosphate buffer adjusted to pH 4.2). The reaction buffer also contained an appropriate concentration of an antibiotic selected from among those common in the industry (e.g. Lactoside, Alltech), 20 mM urea, an appropriate amount of a starch hydrolyzing enzyme (e.g. Stargen, Genencor, Inc.), and an appropriate amount of a yeast (Saccharomyces cerevisiae) strain used for fuel ethanol production (e.g. products from Fleishman's, Lallemand, Inc., LeSaffre Group). Appropriate amounts of enzyme and yeast were provided in the respective manufacturers' instructions.

The vessel was then sealed with a butyl rubber septum and incubated at 30 degrees Centigrade. Data was collected by continuously monitoring the internal pressure of the vessel using a custom fabricated sensor containing a pressure transducer (Jacobsen Holz Corporation, Perry, Iowa).

In order to interpret the pressure data collected across events it was also necessary to perform several activities that allow normalization of data for changes in initial conditions (e.g. changes in sample size, generation of pressure for non-substrate fermentation sources, and changes in initial atmospheric pressure). Atmospheric pressure at the time the reaction vessels were sealed for an event was recorded and data were analyzed with atmospheric pressure as a co-variate. The exact mass of each sample being analyzed was determined precisely and data expressed as gas production/gram of sample. The exact volume of gas produced from non-substrate sources in the reaction was determined by analysis of replicated reaction vessels which contained all other reaction ingredients but without added fermentation substrate (grain sample). The magnitude of the pressure change in these “control” reaction vessels was subtracted from each experimental measurement (each sample) at each time point when data was collected. Thus the gas production reported can be attributed to the presence of the experimental sample and to no other variable.

The changes in pressure were caused by the generation of carbon dioxide from glucose fermentation and the rate of change in pressure was indicative of starch hydrolysis/fermentation rate. Because this pressure change data was collected at many points using a customized computer interface (Jacobsen Holz Corporation, Perry, Iowa) during the fermentation, it was very useful in determining the kinetic properties of the substrate being tested.

### Example 5

### Determination of Amylose/Amylopectin Content and Total Starch

Amylose was determined with a kit (K-AMYL) provided by Megazyme International Ltd., Co. Wicklow, Ireland, based on the following reference: Yun, S.-H and Matheson, N. K. (1990) Estimation of amylose content of starch after precipitation of amylopectin by concanavalin-A. Starch/Starke 42:302-305.

### Total Starch was Measured as Follows:

Samples (e.g., mature grain or endosperm) were ground to fine powder, equilibrated to ambient conditions, and stored at room temperature. For analysis, 100 mg of ground tissue were placed into a 2 ml screw cap eppendorf tube. Added to the tissue was 0.9 mL of MOPS buffer (50 mM MOPS, pH 7.0, 5 mM CaCl2, 0.02% Na-azide) containing 100 units of a heat stable Bacillus licheniformis a-amylase. Also added was a ¼″ stainless steel bearing. Tubes were capped, vortexed, and processed through a Geno/Grinder (Glen Mills, Clifton, N.J.) for 20 sec at 1,650 strokes per minute. They were next rotated (16 rpm) in a oven at 90° C. for 75 minutes. Tubes were removed and processed through the Geno/Grinder as before. After the temperature of the digests was reduced to 55° C., 0.6 mL of acetate buffer (285 mM Na-acetate, pH 4.5, 0.02% Na-azide) containing five units of Aspergillus niger amyloglucosidase (certified to contain only very low levels of b-glucanase activity) was added. Tubes were sealed and incubated at 55° C. with constant rotation (16 rpm) for 15-18 hours. Tubes were then boiled for five minutes, equilibrated to room temperature, mixed, and the bearing removed. Digests were stored frozen. Ahead of determining the concentration of glucose in the digests, the tubes were mixed and centrifuged (14,000×g) for five minutes. Glucose concentration was determined using a Skalar San++ instrument (Skalar, The Netherlands) and a “Glucose/Fructose” method (catnr. 353), which is based on an enzyme catalyzed reaction involving hexokinase and glucose-6-phosphate dehydrogenase. The method was modified for appropriate levels of sample dilution. Two blanks and a set of 12 glucose standards were also processed with each set of samples. For the standards, dry glucose was weighed directly into eppendorf tubes and carried through the entire digestive process and procedure for determination of glucose. Duplicate digests were prepared for all samples and duplicate determinations of glucose were made for each digest. Free sugars were not removed ahead of starch digestion, as repeated studies have shown that the level soluble glucose from maize tissue is negligible compared to starch-derived glucose and the error associated with this method (C.V. <1.0). Results were corrected for moisture content and reported on a dry weight basis.

Measurements for percent amylose were calculated as a mean across 5 transgenic events and compared to null kernels from the same ear. The transgenic waxy kernels generated from PHP23504 had 3.1% amylose/96.9% amylopectin vs 24.3% amylose/75.7% amylopectin in the null (wild-type) kernels.

Other objects, features, advantages and aspects of the present invention will become apparent to those of skill from the following description. It should be understood, however, that the foregoing description and the specific examples, while indicating certain embodiments of the invention, are given by way of illustration only. Various changes and modifications within the spirit and scope of the disclosed invention will become readily apparent to those skilled in the art from reading the description and other parts of the present disclosure.

What is claimed is:

1. An isolated nucleic acid sequence comprising SEQ ID NO:1.

2. An expression cassette comprising a chimeric nucleic acid sequence wherein:
a) a fragment comprising nucleotides 1-280 of SEQ ID NO:1 is operably linked to nucleotides 1561-1827 of SEQ ID NO:1 in sense orientation;
b) the fragment of a) in antisense orientation is operably linked to a spliceable ADH1 intron; and
c) the fragment of (b) is operably linked to the fragment of a).

3. The expression cassette of claim 2 wherein the chimeric nucleic acid sequence is operably linked to a promoter that drives expression in maize seed.

4. A method of increasing the percentage levels of amylopectin starch compared to wild-type levels in maize grain, the method comprising:
a) transforming a maize plant cell with at least one expression cassette comprising the chimeric nucleic acid sequence of claim 2;
b) growing the transformed cell to generate a maize plant seed with a dominant waxy grain genotype; and
c) screening the grain for increased percentage level of amylopectin starch.

5. A method of producing maize grain having increased percentage levels of amylopectin starch compared to wild-type maize, the method comprising:
a) transforming a maize plant cell with at least one expression cassette comprising the chimeric nucleic acid sequence of claim 2; and
b) growing the transformed cell to generate a maize plant having a dominant waxy grain genotype having increased levels of amylopectin starch.

6. A maize plant having the expression cassette of claim 2 stably transformed into its genome, wherein expression of the DNA construct results in maize grain having an increased percentage level over wild-type maize grain of amylopectin starch.

7. The transformed maize seed of the transformed plant of claim 5.

8. A method of screening cereal grain for fermentation efficiency, the method comprising:
a) placing a sample of cereal grain in a sealed reaction vessel containing aqueous buffer, urea, a starch-degrading enzyme, and yeast;
b) incubating the vessel at 30° C. while monitoring and recording as data the internal pressure with a sensor; and
c) collecting and interpreting the data compared to one or more control vessels to determine the fermentation efficiency of the cereal grain.
